(19) United States
(12) Patent Application Publication
(10) Pub. No.: US 2009/0233841 A1
(43) Pub. Date: Sep. 17, 2009

(54) ANALGESIC CONJUGATES

(75) Inventors: Philip S. Portoghese, St. Paul, MN (US); Sandra C. Roerig, Shreveport, LA (US)

Correspondence Address: VIKSNINS HARRIS & PADYS PLLP, P.O. BOX 111098, ST. PAUL, MN, 55111-1098, US

(73) Assignees: Regents of the University of Minnesota, St. Paul, MN (US); The Board of Supervisors of the Louisiana State University and Agricultural and Mechanical College, Shreveport, LA (US)

(21) Appl. No.: 11/813,073

(22) PCT Filed: Jan. 5, 2005
(86) PCT No.: PCT/US05/00181

§ 371 Date: Aug. 5, 2008

(51) Int. Cl. A61K 38/02; A61K 31/485; C07D 491/18; C07K 2/00; A61P 25/04

(52) U.S. Cl. 514/2; 514/282; 546/35; 530/300

(57) ABSTRACT

The present invention relates generally to analgesic compounds having a mu opioid receptor agonist linked to a delta opioid receptor antagonist, and to methods for producing analgesia using such compounds. As compared to opioids such as morphine, these compounds can cause less tolerance, physical dependence, and/or constipation. These compounds are also more potent than morphine and are able to cross the blood brain barrier, thereby allowing for peripheral (e.g., IV) administration.

### STATEMENT REGARDING FEDERALLY SPONSORED RESEARCH OR DEVELOPMENT

Work relating to this application was supported by grants from the National Institutes of Health (DA15091 and DA18028). The United States government may have certain rights in the invention.

### BACKGROUND OF THE INVENTION

Pain represents a major health and economic problem throughout the world. Despite advances in understanding the physiological basis of pain, an ideal analgesic has yet to be discovered.

Among analgesic drugs, the opioid class of compounds is widely used for pain treatment. The opioid drugs produce effects by interacting with the opioid receptors. The existence of at least three opioid receptor types, μ (mu), δ (delta), and κ (kappa) has been established. All three opioid receptor types are located in the human central nervous system, and each has a role in the mediation of pain.

Morphine and related opioids currently used as analgesics produce their analgesia primarily through their agonist action at mu opioid receptors. The administration of these drugs is limited by significant side effects such as the development of tolerance, physical dependence, addiction liability, constipation, respiratory depression, muscle rigidity, and emesis. Accordingly, there is a need for improved analgesics.

### SUMMARY OF CERTAIN EMBODIMENTS OF THE INVENTION

Disclosed herein are analgesic conjugates having a mu opioid receptor agonist linked via a linker to a delta opioid receptor antagonist. Surprisingly, these conjugates typically can cause less tolerance, physical dependence, and constipation than is caused by opioids such as morphine. These conjugates are also typically more potent than morphine and are typically able to cross the blood brain barrier, thereby allowing for peripheral (e.g., intravenous (IV)) administration.

Accordingly, certain embodiments of the present invention provide analgesic conjugates having a mu opioid receptor agonist linked to a delta opioid receptor antagonist, and to methods for producing analgesia using such conjugates.

Some embodiments of the invention provide the use of a conjugate that includes a mu opioid receptor agonist linked via a linker to a delta opioid receptor antagonist to prepare a medicament useful for producing analgesia in a patient following administration at a location outside the central nervous system of the patient.

Some embodiments of the invention provide the use of a conjugate that includes a mu opioid receptor agonist linked via a linker to a delta opioid receptor antagonist to prepare a medicament useful for producing analgesia while causing less inhibition of gastrointestinal (GI) transit than is caused by administration of a similar effective dosage of morphine to the patient.

Some embodiments of the invention provide the use of a conjugate that includes a mu opioid receptor agonist linked via a linker to a delta opioid receptor antagonist to prepare a medicament useful for producing analgesia while causing less dependence than is caused by administration of a similar effective dosage of morphine to the patient.

Some embodiments of the invention provide the use of a conjugate that includes a mu opioid receptor agonist linked via a linker to a delta opioid receptor antagonist to prepare a medicament useful for producing analgesia while causing less tolerance than is caused by administration of a similar effective dosage of morphine to the patient.

Some embodiments of the invention provide a method for producing analgesia in a patient, including administering to the patient an effective amount of a conjugate that includes a mu opioid receptor agonist linked via a linker to a delta opioid receptor antagonist, which administration is to a location outside the central nervous system of the patient.

Some embodiments of the invention provide a method for producing analgesia while causing less inhibition of gastrointestinal (GI) transit than is caused by administration of a similar effective dosage of morphine in a patient, including administering to the patient an effective amount of a conjugate that includes a mu opioid receptor agonist linked via a linker to a delta opioid receptor antagonist effective to cause analgesia while causing less inhibition of GI transit than is caused by administration of a similar effective dosage of morphine to a patient.

Some embodiments of the invention provide a method for producing analgesia while causing less dependence than is caused by administration of a similar effective dosage of morphine in a patient, including administering to the patient an effective amount of a conjugate that includes a mu opioid receptor agonist linked via a linker to a delta opioid receptor antagonist effective to cause analgesia while causing less dependence than is caused by administration of a similar effective dosage of morphine to a patient.

Some embodiments of the invention provide a method for producing analgesia while causing less tolerance than is caused by administration of a similar effective dosage of morphine in a patient, including administering to the patient an effective amount of a conjugate that includes a mu opioid receptor agonist linked via a linker to a delta opioid receptor antagonist effective to cause analgesia while causing less tolerance than is caused by administration of a similar effective dosage of morphine to a patient.

Some embodiments of the invention provide a method for producing analgesia while causing less addiction liability than is caused by administration of a similar effective dosage of morphine in a patient, including administering to the patient an effective amount of a conjugate that includes a mu opioid receptor agonist linked via a linker to a delta opioid receptor antagonist effective to cause analgesia while causing less addiction liability than is caused by administration of a similar effective dosage of morphine to a patient.

Some embodiments of the invention provide a pharmaceutical composition including: a conjugate that includes a mu opioid receptor agonist linked via a linker to a delta opioid receptor antagonist; and a pharmaceutically acceptable carrier other than saline; which composition is formulated for IV administration.

Some embodiments of the invention provide a pharmaceutical composition including: a conjugate that includes a mu opioid receptor agonist linked via a linker to a delta opioid receptor antagonist; and a pharmaceutically acceptable carrier that includes saline and at least one other pharmaceutically acceptable carrier; which composition is formulated for intravenous administration.

Some embodiments of the invention provide a conjugate having the formula:

R₁—X₁—R₂

wherein

R₁ is a mu opioid receptor agonist;

R₂ is a delta opioid receptor antagonist; and

X₁ is a linker, provided that the conjugate is not a conjugate of the formula:

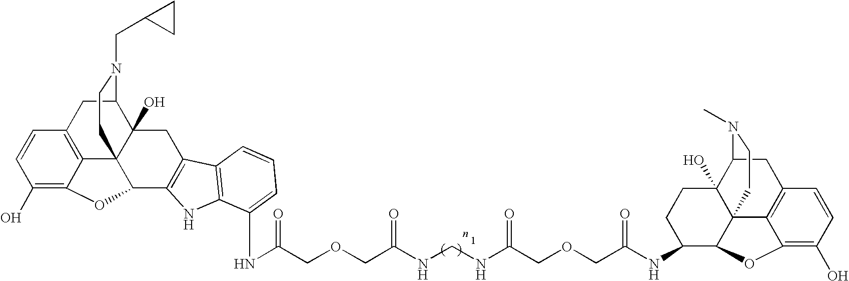

wherein n₁ is 2, 3, 4, 5, 6, or 7.

Some embodiments of the invention provide a conjugate having the formula:

R₁—X₁—R₂

wherein

R₁ is a mu opioid receptor agonist that is not α-oxymorphamine;

R₂ is a delta opioid receptor antagonist; and

X₁ is a linker.

Some embodiments of the invention provide a conjugate having the formula:

R₁—X₁—R₂

wherein

R₁ is a mu opioid receptor agonist;

R₂ is a delta opioid receptor antagonist that is not naltrindole; and

X₁ is a linker.

Some embodiments of the invention provide a conjugate having the formula:

R₁—X₁—R₂

wherein

R₁ is a mu opioid receptor agonist;

R₂ is a delta opioid receptor antagonist; and

X₁ is a linker that is not

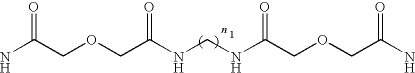

wherein n₁ is 2, 3, 4, 5, 6, or 7.

Some embodiments of the invention provide a pharmaceutical composition including a pharmaceutically acceptable excipient and a conjugate of the invention.

Some embodiments of the invention provide a unit dosage form including a conjugate of the invention and a pharmaceutically acceptable excipient.

Some embodiments of the invention provide a conjugate of the invention for use in medical therapy.

Some embodiments of the invention provide a use of a conjugate of the invention to prepare a medicament for treating pain in an animal.

### BRIEF DESCRIPTION OF THE FIGURE

FIG. 1 depicts the results of a conditioned place preference experiment.

### DETAILED DESCRIPTION OF THE INVENTION

It has been discovered that conjugates having a mu opioid receptor agonist linked via a linker to a delta opioid receptor antagonist are typically analgesic and can typically cause less tolerance, physical dependence, and constipation than is caused by opioids such as morphine. These conjugates are also typically more potent than morphine and are able to typically cross the blood brain barrier, thereby allowing for peripheral (e.g., IV) administration of the conjugates.

A series of conjugates containing a mu opioid receptor agonist linked via a linker to a delta opioid receptor antagonist were designed, synthesized and evaluated by intracerebroventricular (ICV) administration. The conjugate with the shortest linker produced both tolerance and dependence with chronic ICV administration that was similar to both morphine and a control ligand. However, when the distance between the two pharmacophores (i.e., the mu opioid receptor agonist and the delta opioid receptor antagonist) of the conjugate exceeded 22 Å, the conjugate no longer produced tolerance or dependence. While not necessarily an element of any specific embodiment of the invention, it is believed that the conjugates with longer linkers interact simultaneously with neighboring mu and delta opioid receptors.

Pretreatment with the delta antagonist naltrindole altered the ED₅₀ values of the conjugate MDAN-19 so that its acute analgesic activity was similar to the control ligand MA-19. This effect was not observed for MDAN-16. These data suggest that a physical interaction between the mu and delta opioid receptors modulate tolerance and dependence and that the conjugates MDAN-19, MDAN-20, and MDAN-21 hold the dimerized receptors together, thereby preventing reorganization.

Some embodiments of the invention provide a method for producing analgesia while causing less inhibition of gastrointestinal (GI) transit, less dependence, less tolerance, less addictive liability, and/or less constipation than is caused by administration of a similar effective dosage of a mu opioid receptor agonist in a patient, including administering to the patient an effective amount of a conjugate that includes the mu opioid receptor agonist linked via a linker to a delta opioid receptor antagonist effective to cause analgesia while causing less inhibition of GI transit, less dependence, less tolerance, less addictive liability, and/or less constipation than is caused by administration of a similar effective dosage of the mu opioid receptor agonist to a patient.

In some embodiments of the invention, the administration of the effective amount of the conjugate causes less inhibition of gastrointestinal (GI) transit than is caused by administration of a similar effective dosage of morphine to a patient.

In some embodiments of the invention, the administration of the effective amount of the conjugate causes less constipation than is caused by administration of a similar effective dosage of morphine to a patient.

In some embodiments of the invention, the administration of the effective amount of the conjugate causes less dependence than is caused by administration of a similar effective dosage of morphine to a patient.

In some embodiments of the invention, the administration of the effective amount of the conjugate causes less tolerance than is caused by administration of a similar effective dosage of morphine to a patient.

In some embodiments of the invention, the administration of the effective amount of the conjugate causes less addiction liability than is caused by administration of a similar effective dosage of morphine to a patient.

In some embodiments of the invention, the administration is to a location outside the central nervous system of the patient. In some embodiments of the invention, the administration is intravenous. In some embodiments of the invention, the administration is intrathecal.

In some embodiments of the invention, the conjugate has the formula:

R₁—X₁—R₂

wherein

R₁ is a mu opioid receptor agonist;

R₂ is a delta opioid receptor antagonist; and

X₁ is a linker.

In some embodiments of the invention, X₁ includes an amino acid. In some embodiments of the invention, X₁ includes a peptide.

In some embodiments of the invention, X₁ is a divalent, branched or unbranched, saturated or unsaturated, hydrocarbon chain, having from 10-30 carbon atoms in the chain, wherein one or more of the carbon atoms in the chain is optionally replaced by (—O—) or (—NH—).

In some embodiments of the invention, X₁ is a divalent, branched or unbranched, saturated or unsaturated, hydrocarbon chain, having from 10-30 carbon atoms in the chain, wherein one or more of the carbon atoms in the chain is optionally replaced by (—O—) or (—NH—), and wherein the chain is optionally substituted on at least one carbon, —O— or —NH— with one or more substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

In some embodiments of the invention, X₁ is a chain 18-24 atoms in length. In some embodiments of the invention, X₁ includes a central diamine moiety having adjacent diglycolic acid molecules. In some embodiments of the invention, X₁ includes at least one methylene.

In some embodiments of the invention, R₁ is oxymorphone, α-oxymorphamine, a benzomorphan, etonitazine, fentanyl, or a compound of formula 100, 101, 102, 103, 104, or a derivative thereof.

In some embodiments of the invention, R₂ is naltrindole or a compound of formula 201, 202, or 203, or a derivative thereof.

In some embodiments of the invention, the conjugate has the following formula:

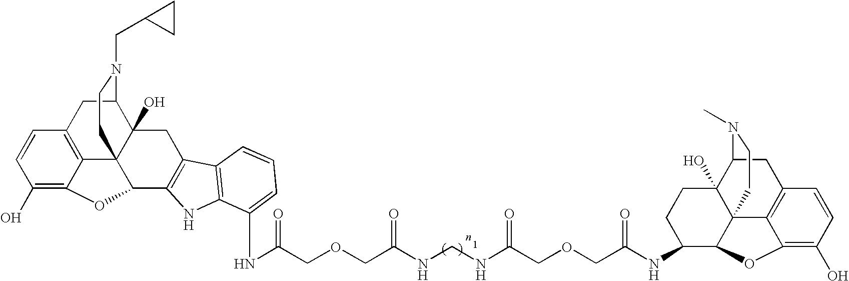

wherein n₁ is 1, 2, 3, 4, 5, 6, 7, 8, 9, or 10. In some embodiments of the invention, n₁ is 5, 6, or 7.

In some embodiments of the invention, the conjugate is administered in combination with at least one additional therapeutic agent.

In some embodiments of the invention, the conjugate is not a conjugate of the formula:

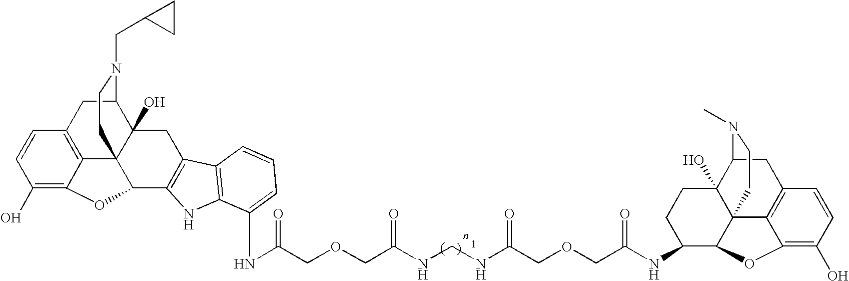

wherein n₁ is 2, 3, 4, 5, 6, or 7.

In some embodiments of the invention, R₁ is a mu opioid receptor agonist that is not α-oxymorphamine. In some embodiments of the invention, R₁ is a benzomorphan, etonitazine, fentanyl, or a compound of formula 100, 101, 102, 103, 104, or a derivative thereof.

In some embodiments of the invention, R₂ is a delta opioid receptor antagonist that is not naltrindole. In some embodiments of the invention, R₂ is derivative or naltrindole or a compound of formula 201, 202, or 203, or a derivative thereof.

In some embodiments of the invention, X₁ is a linker that is not

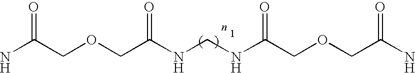

wherein n₁ is 2, 3, 4, 5, 6, or 7.

The analgesic conjugates will be useful as analgesics, typically with limited side effects, for example, analgesics that typically lead to the development of relatively less tolerance, physical dependence, and constipation as compared to the development of those side effects from a similarly effective analgesic dose of morphine or from a similarly effective analgesic dose of the mu opioid agonist. It should be understood that any linker can be used that can hold the pharmacophores apart, for example, a linker of greater than the minimum distance (e.g., greater than about 16 atoms). This length is thought to be important for bridging the recognition sites of the mu and delta opioid receptors. In addition, the linkers may confer or maintain a favorable hydrophilic-lipophilic balance for access into the central nervous system (CNS) upon administration to a location outside of the CNS, e.g., via oral or parenteral administration. For example, the linker may have hydrophobic and hydrophilic groups in repeating units so that lengthening the linker would not substantively alter the balance of the conjugate.

The analgesic conjugates thus contain a delta opioid receptor antagonist pharmacophore linked through varying length linkers to a mu opioid receptor agonist pharmacophore. Combinations of delta antagonist pharmacophores and mu agonist pharmacophores are presented herein, and other combinations of pharmacophores could also be employed.

Several linkers are presented herein, and there are numerous other possible linkers that could also be used. The linkers can be homologated to afford the conjugate with a particular combination of pharmacophores. Regioisomers and stereoisomers of the pharmacophores also may be used, e.g., due to the NH substitution on the pharmacophores.

Mu Opioid Receptor Agonists A “mu opioid receptor agonist” refers to any compound that binds to a mu opioid receptor, e.g., selectively binds to a mu opioid receptor, and activates the mu opioid receptor. The ability of a compound to act as a mu opioid receptor agonist may be determined using pharmacological methods well known in the art.

Mu opioid receptor agonists include, but are not limited to, oxymorphone, α-oxymorphamine, benzomorphans, etonitazine, fentanyl,

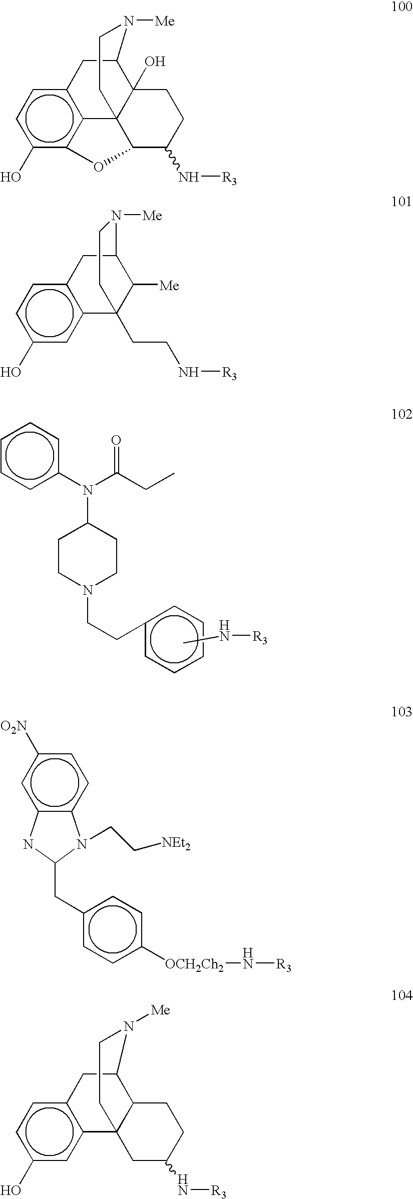

and derivatives thereof, wherein R₃ above represents one possible point of attachment for a linker linked to a delta opioid receptor antagonist. Also, see Foye's Principles of Medicinal Chemistry, 5th Ed., D. A. Williams and T. L. Lemke, Eds, Lippencott, Williams, and Wilkins, and especially Chapter 19, including pages 462-465.

Delta Opioid Receptor Antagonists A “delta opioid receptor antagonist” refers to any compound that attenuates the effects of a delta opioid receptor agonist. The ability of a compound to act as a delta opioid receptor antagonist may be determined using pharmacological methods well known in the art.

Delta opioid receptor antagonists include, but are not limited to those delta opioid receptor antagonists disclosed in U.S. Pat. Nos. 6,271,239; 5,631,263; 5,578,725; 5,464,841; 5,411,965; 5,352,680; and 4,816,586 and in Daniels et al., “Delta-Selective Ligands Related to Naltrindole”, in “The Delta Receptor”, Chang et al., Eds. Marcel Dekker, Chapter 9, pages 139-158 (2003). Delta opioid receptor antagonists also include, but are not limited to naltrindole,

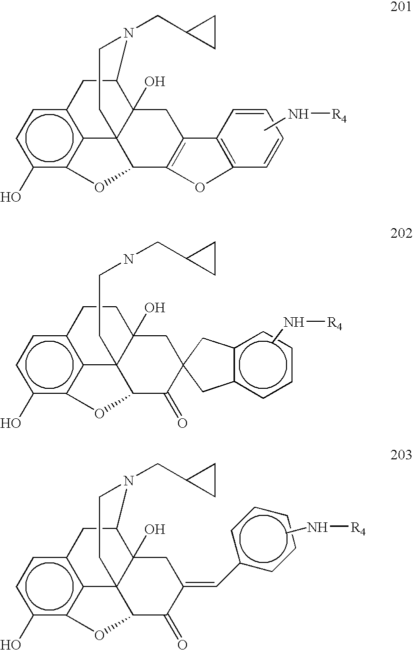

and derivatives thereof, wherein R₄ above represents one possible point of attachment for a linker linked to a mu opioid receptor agonist.

Linkers The conjugates of the invention have a mu opioid receptor agonist linked via a linker to a delta opioid receptor antagonist. The attachment of the linker to the pharmacophores, e.g., the point of attachment, should not eliminate the pharmacophores' activity as a mu opioid receptor agonist or as a delta opioid receptor antagonist. In some embodiments of the invention, the linker has a central diamine moiety with adjacent diglycolic acid molecules. The linker length may be varied in certain embodiments of the invention by increasing or decreasing the number of atoms in the linker (e.g., by varying the number of methylenes in the central diamine portion). In some embodiments of the invention, the linkers can be constructed to establish and/or maintain a favorable hydrophilic-hydrophobic balance of the conjugate. In some embodiments of the invention, the linkers vary from 16 atoms (e.g., MDAN-16) to 21 atoms (e.g., MDAN-21).

In some embodiments of the invention, the linker length is about 16, 17, 18, 19, 20, 21, 22, 23, 24, or 25 Å long. In some embodiments of the invention, the linker length is from about 22 to 26 Å. In some embodiments of the invention, the linker length is 16, 17, 18, 19, 20, 21, 22, 23, 24, or 25 atoms long. In some embodiments, the linker is 18-24 atoms long. The art worker can calculate the length of a specific linker using molecular modeling software available to the art worker, for example, using Chem3D Pro 9.0 (CambridgeSoft Corporation).

Linkers may also contain amino acids, peptides, and glycolic acids.

The term “amino acid,” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also includes natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M. “Protecting Groups In Organic Synthesis” second edition, 1991, New York, John Wiley & sons, Inc., and references cited therein).

The term “peptide” describes a sequence of 2 to 35 amino acids or peptidyl residues. The sequence may be linear or cyclic. For example, a cyclic peptide can be prepared or may result from the formation of disulfide bridges between two cysteine residues in a sequence. Peptide derivatives can be prepared, for example, as disclosed in U.S. Pat. Nos. 4,612,302; 4,853,371; and 4,684,620.

In some embodiments of the invention, the exact nature of the linker in a conjugate is not critical. The linker is in some embodiments a divalent organic radical having a molecular weight of from about 25 daltons to about 400 daltons. The linker in some embodiments has a molecular weight of from about 40 daltons to about 200 daltons.

The linker may be biologically inactive, or may itself possess biological activity. The linker can also include other functional groups (including hydroxy groups, mercapto groups, amine groups, carboxylic acids, as well as others) that can be used to modify the properties of the conjugate (e.g. for branching, for cross linking, for appending other molecules (e.g. a biologically active compound) to the conjugate, for changing the solubility of the conjugate, or for effecting the biodistribution of the conjugate).

Specific values listed herein for radicals, substituents, groups, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

In some embodiments, the linker is a divalent, branched or unbranched, saturated or unsaturated, hydrocarbon chain, having from 1 to 50, e.g, from 10 to 30, e.g., from 20 to 30, carbon atoms, wherein the chain is optionally substituted on at least one carbon atom with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy. One or more of the carbon atoms may be optionally replaced by another atom such as (—O—) or (—N—). The chain may also be optionally substituted on at least one carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

In some embodiments of the invention, the linker may be:
CO—CH₂OCH₂CO—NH—(CH₂)ₙ₂NH—CO—CH₂OCH₂CO;
CO—CH₂O(CH₂CH₂O)ₙ₃CH₂CO;
(CO—CH₂NH)ₓ₂—CO—(CH₂)ₙ—CO—(NH—CH₂CO)_{X2};
CO—(CH₂)ₙ₃CO; or

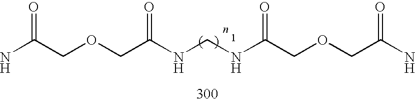

wherein n₁ is any integer from 1-10, n₂ is any integer from 1-8, n₃ is any integer from 1-20, n₄ is any integer from 12-22, and x2 is any integer from 1-2.

Opioid Conjugates An opioid conjugate is a compound that has a mu opioid receptor agonist linked via a linker to a delta opioid receptor antagonist. The conjugate thus contains two different pharmacophores linked together (i.e., a mu opioid receptor agonist linked to a delta opioid receptor antagonist).

The nomenclature used herein to name compounds is as follows: M=a μ (mu) pharmacophore, D=a δ (delta) pharmacophore, A=agonist, N=antagonist. The digits refer to the number of atoms in the linker. For example, MDAN-21 refers to a conjugate having a mu opioid receptor agonist linked via a linker including a chain of 21 atoms to a delta opioid receptor antagonist. MA-21 refers to a compound having a mu opioid receptor agonist having a linker of 21 atoms. DN-20 refers to a compound having a delta opioid receptor antagonist having a linker of 20 atoms.

Specific opioid conjugates are disclosed herein, including MDAN-16, MDAN-17, MDAN-18, MDAN-19, MDAN-20, and MDAN-21 (compounds 3-8, respectively). Other opioid conjugates can be synthesized by linking a mu opioid receptor agonist to a delta opioid receptor antagonist.

Analgesia Analgesia refers to a state in which a painful stimulus elicits a decreased sensation of pain as compared to the pain sensation elicited without analgesia. Analgesic effectiveness of drugs in humans is predicted by the tail flick test (Hammond, 1989), and can be evaluated using methods known to the art worker, e.g., using the radiant heat tail flick assay (D'Amour et al., 1941). Briefly, in the radiant heat tail flick assay, a beam of light is focused on a mouse tail and the time until the tail flicks is measured. Each animal may serve as its own control and may be used only once. Mice are tested once before injection of a drug (control time). After injection of the drug, the mice are tested at the time of peak drug response (drug time). The light intensity can be adjusted so that control times are between 1.5 and 2.5 seconds. A 10 second cutoff drug time can be set to minimize the risk of tissue damage. Percent maximum possible effect (% MPE) is calculated as follows (Dewey et al., 1970):

$$\frac{Drug Time ( s ) - Control Time ( s )}{10 seconds - Control Time ( s )} \times 100 \% = \% MPE$$

Graded dose response curves of at least 4 doses with at least 8 mice per dose can be generated from the % MPE data. ED50 values with 95% confidence intervals can be computed with GraphPad Prism using nonlinear regression methods.

In certain embodiments of the invention, the conjugates are used to cause analgesia to treat pain, e.g., acute and/or chronic pain. In some embodiments of the invention, the conjugates cause their analgesic effects in the central nervous system. Thus, the ability of the conjugates to pass through the blood brain barrier to allow for IV administration is advantageous.

Addiction, Dependence, and Tolerance Addiction refers to the development of dependence, e.g., physical dependence, on a drug. Development of tolerance to a drug may occur so that when a drug has been administered for a period of time, the same dosage of that drug produces less of an effect, thereby leading to the need for increasing the dosage of that drug to achieve the same effect, e.g., the same amount of analgesia. Physical dependence on a drug may also occur when a drug has been administered for a period of time and the drug administration is terminated, leading to symptoms of drug withdrawal. Addiction to a drug can occur after chronic drug administration. Addiction liability refers to the potential of a drug to be abused for its rewarding properties, e.g., to the presumed preference of a subject to a specific drug so that a subject will prefer to remain in an environment associated with use of that drug.

To investigate the addiction, physical dependence, and tolerance associated with a compound, chronic ICV administration studies can be performed in mice. The compound of interest can be administered ICV via a cannula using an osmotic minipump for a period of time, e.g., for 3 days, as previously described (Gomori et al., 2003; Mashiko et al., 2003). Withdrawal can be measured by administering naloxone (1 mg/kg; subcutaneous (sc)) and counting the naloxone-precipitated jumps for 10 minutes. Tolerance can be determined by administering challenge dosed of the compound after chronic infusion for 3 days, and determining the chronic ED₅₀ value at that time. (see Ho et al., 1972; Way, 1978; Kest et al., 1996; and Van der Kooy et al., 1982).

The conditioned place preference (CPP) test is a technique used to measure the rewarding properties and addiction liability of a drug. Briefly, two sides of a CPP apparatus may have both visual and tactile differences, so that an animal can distinguish between the sides. On the first day of testing, the time each animal spends in either side of the apparatus is measured. For the next three days, a drug is “paired” with one side or the other by injecting the animal and immediately confining it to that side. On the final day, the amount of time the animal spends in the drug-paired side is determined and the percent change is calculated. If the percent change is positive, the drug is rewarding and presumed to be addictive. (see Bardo et al., 2000; and Wu et al., 2004)

Constipation Constipation can be caused by inhibition, e.g., opioid-induced inhibition, of gastrointestinal (GI) transit. To evaluate the effect a drug has on GI transit, the drug can administered IV via the tail vein, e.g., in a volume of 100 μl to mice. Fifteen minutes later, charcoal meal (300 μl, oral) can be administered by gavage. Thirty minutes after the charcoal meal, the mice can be sacrificed by halothane overdose and the distance the charcoal traveled relative to the entire length of the GI tract can be compared to the distance traveled in control animals injected with saline. A drug that inhibits GI transit will decrease the distance the charcoal travels and will cause constipation.

Potency The analgesic potency of a compound can be determined by methods known to the art worker, e.g., the potency may be determined by the tail flick assay.

In some embodiments of the invention, the conjugates are at least about as potent as morphine; are at least about 10 times as potent as morphine, at least about 50 times as potent as morphine, or at least about 100 times as potent as morphine.

In cases where conjugates are sufficiently basic or acidic to form stable nontoxic acid or base salts, administration of the conjugates as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The conjugates can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Thus, the conjugates may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the conjugate may be combined with one or more excipients and used in the form of ingestible tablets, buccal tablets, troches, capsules, elixirs, suspensions, syrups, wafers, and the like. Such compositions and preparations may contain at least 0.1% of active compound. The percentage of the compositions and preparations may, of course, be varied and may conveniently be between about 2 to about 60% of the weight of a given unit dosage form. The amount of conjugate in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The conjugate may also be administered intravenously, intraarterially, or intraperitoneally by infusion or injection. Solutions of the conjugate or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders including the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium including, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin. In some embodiments of the invention, the pharmaceutical dosage contains a pharmaceutically-acceptable carrier other than saline, and in some embodiments does not contain saline. In some embodiments of the invention, the pharmaceutical dosage contains saline and at least one other pharmaceutically-acceptable carrier.

Sterile injectable solutions are prepared by incorporating the conjugate in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present conjugates may be applied in pure form. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the conjugates can be determined by comparing their in vitro activity, and in vivo activity in animal models. Methods for the extrapolation of effective dosages in mice, and other animals, to humans are known to the art; for example, see U.S. Pat. No. 4,938,949.

In some embodiments, the concentration of the conjugate in a liquid composition, such as a lotion, will be from about 0.1-25 wt-%, preferably from about 0.5-10 wt-%. The concentration in a semi-solid or solid composition such as a gel or a powder can be about 0.1-5 wt-%, preferably about 0.5-2.5 wt-%.

The amount of the conjugate, or an active salt or derivative thereof, required for use in treatment will vary not only with the particular conjugate selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g. into a number of discrete loosely spaced administrations; such as multiple inhalations from an insufflator or by application of a plurality of drops into the eye.

One or more of the conjugates can be administered by any route appropriate for the condition being treated. Suitable routes include transdennal, oral, rectal, nasal, topical, buccal, sublingual, vaginal, parenteral, subcutaneous, intramuscular, intravenous, intraarterial, intradermal, intrathecal, epidural, and the like. An advantage of the conjugates presented herein is that they typically can be administered to a location outside of the central nervous system, e.g., I.V., so as to have their analgesic effects centrally.

Methods of Making the Compounds of the Invention The invention also relates to methods of making the conjugates and compositions of the invention. The compositions are prepared by any of the applicable techniques of organic synthesis. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., Advanced Organic Chemistry, Third Edition, (John Wiley & Sons, New York, 1985), Comprehensive Organic Synthesis. Selectivity Strategy & Efficiency in Modern Organic Chemistry. In 9 Volumes, Barry M. Trost, Editor-in-Chief (Pergamon Press, New York, 1993 printing).

A number of methods for the preparation of the conjugates and compositions of the invention are provided herein. These methods are intended to illustrate the nature of such preparations are not intended to limit the scope of applicable methods.

The invention will now be illustrated by the following non-limiting Examples.

### Example 1

### Analgesia with Reduced Tolerance and Dependence

A series of conjugates containing a mu opioid receptor agonist and a delta opioid receptor antagonist were designed, synthesized and evaluated by chronic ICV administration. The shortest compound produced both tolerance and dependence with chronic ICV administration that was similar to both morphine and a control ligand. However, when the distance between the two pharmacophores of the conjugate exceeded 16 atoms, the conjugate no longer produced tolerance or dependence.

Acute studies. For the MDAN series, increasing the linker length led to increased acute antinociceptive potency (ICV). As this profile was not observed for members of the mu control series (MA-16, MA-17, MA-18, MA-19, MA-20, MA-21; see Table 1), suggesting that another factor, such as bridging dimeric mu-delta opioid receptors, may be responsible. The observation that the conjugates were less potent than their control counterparts not having both a mu agonist and a delta antagonists suggests some type of negative modulation with the delta antagonist pharmacophore and the delta opioid receptor. However, this effect is only seen when the two pharmacophores are linked together. Specifically, when a mu agonist compound (MA-19) and delta antagonist pharmacophore (DN-20) were coadministered, the antinociceptive effect was similar to MA-19 when given alone.

TABLE 1
Antinociceptive Potency of Conjugates in the Mouse Tail
Flick Assay after ICV Administration

| | Linker | | | Ligand |
|---|---|---|---|---|
| | Length | ED₅₀ nmol^{a} | | ED₅₀ pmol^{a} |
| Compound | Å | (95% C.L.) | Compound | (95% C.L.) |
| 3 | 19.1 | 1.79 (1.54-2.04) | 9 | 39.39 |
| (MDAN-16) | | | (MA-16) | (31.95-46.83) |
| 4 | 20.4 | 1.49 (1.04-1.95) | 10 | 39.69 |
| (MDAN-17) | | | (MA-17) | (32.94-46.43) |
| 5 | 21.6 | 0.95 (0.68-1.23) | 11 | — |
| (MDAN-18) | | | (MA-18) | |
| 6 | 22.9 | 0.43 (0.36-0.50) | 12 | 39.45 |
| (MDAN-19) | | | (MA-19) | (23.35-49.56) |
| 7 | 24.1 | 0.17 (0.15-0.19) | 13 | 37.25 |
| (MDAN-20) | | | (MA-20) | (29.15-45.34) |
| 8 | 25.4 | 0.08 (0.06-0.10) | 14 | 43.56 |
| (MDAN-21) | | | (MA-21) | (38.94-48.19) |
| MA-19 + | 22.9, | .037 (.031-.043) | | |
| DN-20 | 24.1 | | | |

^{a}ED₅₀ values were calculated as follows: Percent maximum possible effect (% MPE) equals ([Drug time (s) − Control time (s)]/[10 s − Control time (s)]) × 100%. Graded dose response curves of 4 doses with at least 8 mice per dose were generated from the % MPE and % Inhibition data. ED₅₀ values with 95% confidence levels were computed with GraphPad Prism using nonlinear regression methods.

Chronic studies. MDAN-16 was the only conjugate that produced both tolerance and dependence when chronically administered that was similar to the control ligand MA-19. The next two longer compounds, MDAN-17 and MDAN-18, produced tolerance without producing dependence. When the linker length was longer than about 22 Å (e.g., 6-8; MDAN-19, MDAN-20, and MDAN-21), no tolerance or dependence was observed.

A conjugate having optimal linker length may selectively bind to mu-delta heterodimers. Therefore, the binding of the delta antagonist pharmacophore of the conjugate to the delta receptor of a mu-delta heterodimer may attenuate the mechanism that results in the development of tolerance and/or dependence. So, the control compounds not having both the mu agonist and the delta antagonist and the conjugates with shorter linkers (e.g., MDAN-16 and MDAN-18) may be unable to elicit such an effect and in fact may preferentially bind to a different population of opioid receptors that may utilize different signal transducers. One explanation for this is that MDAN-16 and the mu ligands preferentially bind to mu-mu homodimers. MDAN-17 and MDAN-18 may interact with mu-delta heterodimers in a fashion that is different than MDAN-19, MDAN-20 and MDAN-21 because of their shorter linkers. The longer compounds may have additional binding options available, e.g., inter- and intra-dimeric bridging between associated mu-delta receptors. The signal transduction pathways that are available for these receptors may be different depending on the organization of the receptors.

One of the major drawbacks of using morphine to treat chronic pain is the development of both tolerance and dependence with morphine use. The analgesic conjugates presented herein will be of clinical importance because some of the conjugates unexpectedly do not produce tolerance or dependence. Additionally and also unexpectedly, MDAN-21 and morphine have similar ICV to IV ratios (Table 2). This indicates that conjugates can penetrate the blood brain barrier in a similar fashion to morphine. These conjugates represent a novel drug therapy that produces analgesia without the tolerance or dependence that are commonly seen with mu agonists like morphine and fentanyl. Thus, while maintaining the ability to cross the blood brain barrier, these compounds would be efficacious in treating chronic pain syndromes without the risk of developing dependence and without the need of continuously switching drugs because of tolerance.

TABLE 2
ICV and IV ED₅₀s

ED₅₀

| Compound | ICV (nmol) | IV (nmol) |
|---|---|---|
| 8 (MDAN-21) | 0.08 (0.06-0.10) | 3.3 (3.0-3.6) |
| 12 (MA-19) | 0.04 (0.03-0.05) | 1.61 (1.29-1.92) |
| Morphine | 4.1 (3.7-4.8) | 168 (146-178) |

### Results

Rationale for conjugate design and chemistry. The pharmacophores chosen for the MDAN series incorporate the mu opioid agonist α-oxymorphamine 1, and the delta opioid antagonist naltrindole 2 (NTI), linked together through a variable length linker. The pharmacophores were selected because of the relative affinity and selectivity of the parent ligands (see, e.g., Abdelhamid et al., 1991).

The linker features a central diamine moiety with adjacent diglycolic acid molecules. Linker length was varied by increasing or decreasing the number of methylenes in the central diamine portion. The linkers were constructed in an effort to maintain a favorable hydrophilic-hydrophobic balance of the conjugates. The linkers varied from 16-atoms (MDAN-16, 3) to 21-atoms (MDAN-21, 8). Matched control compounds were synthesized for the mu series (MA-9-MA-14) along with a delta control (DN-20) in an effort to factor out possible effects of the linker on activity in the conjugates.

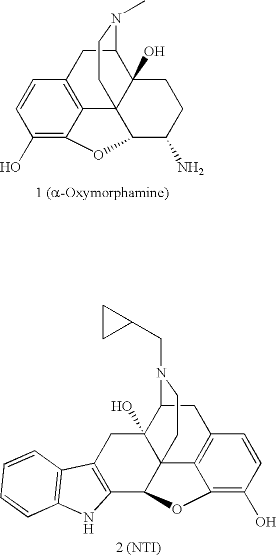

α-Oxymorphamine (1) and the 7′-amino derivative of naltrinidole (2) were the intermediates used. The 7′-amino group of NTI does not radically change its selectivity or potency, and the amino groups of both pharmacophores served as a point of attachment for the linker. The first step was DCC mediated coupling of the Cbz protected diamine linkers 16-21 with 1 followed by deprotection using catalytic transfer hydrogenation to afford intermediates 22-27. The naltrindole intermediate 28 was prepared in a similar fashion by coupling 20 with 7′-Amino-NTI 7′-Amino-NTI and methyl amine were condensed with diglycolic anhydride to give compounds 29 and 30 respectively.

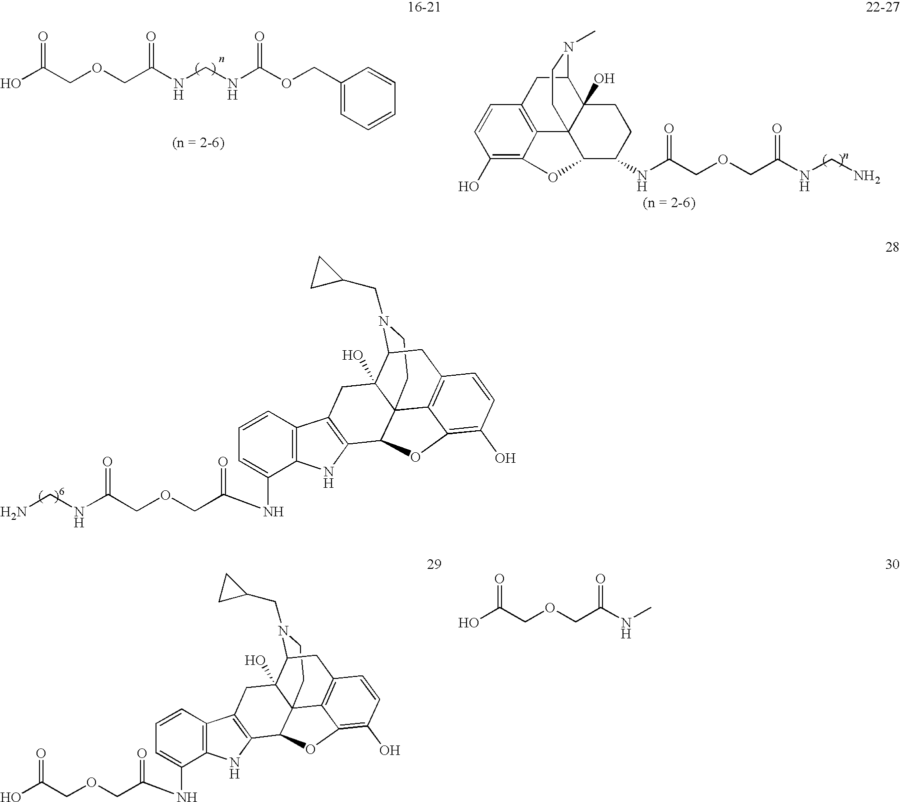

The α-oxymorphamine intermediates 22-27 were subsequently condensed with 28 using DCC to give the final conjugates MDAN-16-MDAN-21, compounds 3-8. Mu analogs 9-14 and delta control 15 were prepared in a similar fashion using the N-methyl analog 30 instead of the second pharmacophore.

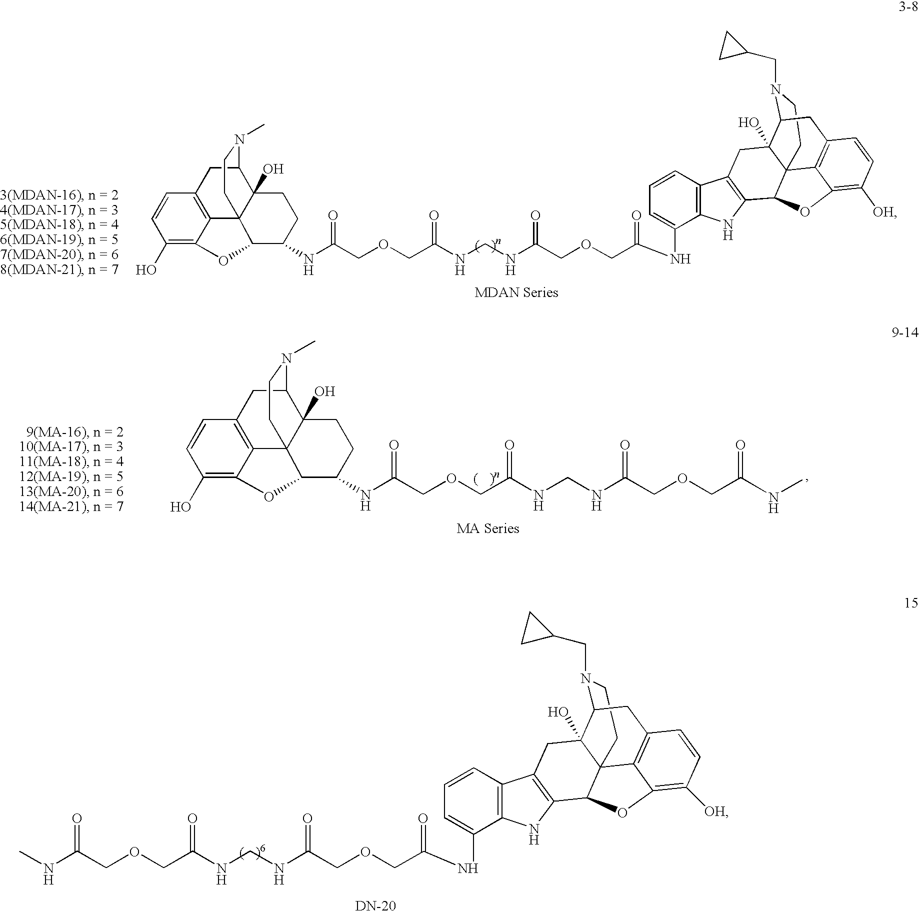

### Pharmacological Results

Acute administration To determine whether linker length affected the potency of the conjugate series, the compounds were acutely administered ICV to mice. The conjugates were less potent than the mu ligands (see Table 1). Increasing the distance between the mu and delta pharmacophores of the MDAN conjugates increased antinociceptive potency, i.e., as the linker length increased, so did the antinociceptive potency (Table 1). In contrast, acute administration of the mu control compounds indicated that the linker length did not affect the potency of these compounds. Coadministration of a mu ligand (MA-19) and delta ligand (DN-20) produced an antinociceptive effect that was similar to MA-19 administered alone (Table 1).

Chronic administration To investigate tolerance and dependence associated with these conjugates, chronic ICV administration studies were performed. The compound of interest was administered ICV via a cannula using an osmotic minipump for 3 days, as previously described (Gomori et al., 2003; Mashiko et al., 2003). Withdrawal was measured by administering naloxone (1 mg/kg; subcutaneous (sc)) and counting the naloxone-precipitated jumps for 10 minutes. Tolerance was determined by administering challenge doses of the compound after chronic infusion for 3 days, and determining the chronic ED₅₀ value at that time. The tolerance and withdrawal properties are summarized in Table 3.

TABLE 3
Tolerance and Naloxone-Precipitated Withdrawal^{a}

| | Saline^{b} ED₅₀ nmol | Chronic^{c} ED₅₀ nmol | | |
|---|---|---|---|---|
| Compound | (95% C.L.) | (95% C.L.) | Difference^{d} | # of Jumps^{e} (SEM) |
| 3 | 1.62 (1.35-1.89) | 4.72 (3.47-5.91) | 2.8 | 30 (23) |
| (MDAN-16) | | | | |
| 4 | 1.54 (0.89-2.20) | 5.61 (4.39-6.83) | 3.6 | 0.9 (0.7) |
| (MDAN-17) | | | | |
| 5 | 1.29 (0.97-1.61) | 4.75 (3.50-6.00) | 3.7 | 8.9 (3.0) |
| (MDAN-18) | | | | |
| 6 | 0.42 (0.37-0.47) | 0.40 (0.33-0.47) | 1.0 | 3.6 (1.7) |
| (MDAN-19) | | | | |
| 7 | 0.17 (0.15-0.20) | 0.17 (0.13-0.21) | 1.0 | 0.4 (0.4) |
| (MDAN-20) | | | | |
| 8 | 0.10 (0.09-0.11) | 0.10 (0.09-0.11) | 1.0 | 3.5 (1.7) |
| (MDAN-21) | | | | |
| 12 | 0.04 (0.03-0.05) | 0.22 (0.19-0.26) | 5.5 | 83 (13) |
| (MA-19) | | | | |
| 12 + 15 | 0.04 (0.03-0.04) | 0.03 (0.02-0.05) | 9.4 | 29 (8) |
| (DN-20) | | | | |
| Morphine | 4.54 (3.51-5.56) | 26.80 (20.82-32.78) | 6.0 | 100 (15) |

^{a}ED₅₀ values were calculated as follows: Percent maximum possible effect (% MPE) equals ([Drug time (s) − Control time (s)]/[10 s − Control time (s)]) × 100%. Graded dose response curves of 4 doses with at least 8 mice per dose were generated from the % MPE and % Inhibition data. ED₅₀ values with 95% confidence levels were computed with GraphPad Prism using nonlinear regression methods.
^{b}Saline was chronically administered ICV for 3 days. Challenge doses were administered after day 3 and the ED₅₀ value was determined. These values are nearly identical to naïve ED₅₀ values (acute administration).
^{c}The compound was chronically administered ICV for 3 days. Challenge doses were administered after day 3 and the ED₅₀ value was determined.
^{d}The difference between the chronic ED₅₀ value and saline ED₅₀ value was calculated.
^{e}Average number of jumps in 10 minutes after administration of naloxone.

Chronic ICV infusion with MDAN-16 produced a 2.6-fold increase in its acute ED50 value, and naloxone induced 30 vertical jumps (Table 3). Thus, both tolerance and physical dependence developed to MDAN-16, the conjugate with the shortest distance between the two pharmacophores. A three-day ICV infusion of MDAN-17 produced a 3.6-fold increase in its ED50 value. In these same mice, naloxone induced an average of only 0.94 vertical jumps (Table 3). MDAN-18 produced results similar to MDAN-17, with a 3.7-fold increase in its acute ED50 value, and only 8.9 naloxone-induced jumps (Table 3). Thus, it appeared that tolerance without physical dependence developed to both MDAN-17 and MDAN-18, both conjugates with intermediate linker lengths.

When mice were infused with MDAN-19, -20, or -21, the acute drug dose response curves were not shifted, and naloxone administration did not induce a significant number of vertical jumps (Table 3). Remarkably, the compounds with longer linkers (MDAN-19, -20, -21) produced no tolerance or physical dependence when administered continuously ICV for three days.

Control experiments were performed in which animals were continuously infused ICV with saline. These animals responded to MA-19, MA-19+DN-20, and the MDAN conjugates similarly to naïve animals in the tail flick assay, i.e., the respective ED50 values were not significantly different (Table 2). Continuous infusion of one mu agonist (MA-19) produced a rightward shift in its acute dose response curve (5.5-fold increase in the ED50). In these same mice, the administration of the nonselective opioid antagonist naloxone precipitated withdrawal as these mice jumped vertically an average of 83 times in a 10-min period after naloxone administration (Table 3). This number of jumps is comparable to that seen in mice infused with morphine for three days (100±15). Coadministration of the mu agonist (MA-19) and the delta antagonist (DN-20) in a 1:1 equimolar ratio produced an 8.9-fold increase in the acute ED50 value; naloxone administration induced 29 vertical jumps in these mice. Thus, MA-19, in the presence or absence of the DN ligand, produced both antinociceptive tolerance and physical dependence. However, it appeared that the degree of physical dependence developed was lesser, and the tolerance developed was greater, in the presence of the delta antagonist (Table 3).

The potency, as determined by the tail flick assay, of morphine, MDAN-21 and control ligand MA-19 was determined by IV administration. All the compounds were more potent when administered ICV (Table 2). Unexpectedly, MDAN-21 had a similar IV to ICV profile to morphine, i.e., there was ˜40× decrease in potency for both compounds when administering IV. Additionally, MDAN-21 was 50-fold more potent than morphine when given IV.

Experimental Procedures

General. All reactions involving moisture sensitive reagents were conducted in oven-dried glassware under nitrogen atmosphere. Solvents were dried when necessary. All other chemicals and solvents were reagent grade unless specified and were obtained from Aldrich Chemical Company, Milwaukee, Wis. ¹H NMR Spectra were recorded on a Varian 300 MHz spectrometer referenced to the solvent. Chemical shifts are expressed in ppm and, coupling constants (J) are expressed in hertz (Hz). Peak multiplicities are abbreviated: broad, br; singlet, s; doublet, d; triplet, t; quartet, q; pentet, p, and multiplet, m. Fast-atom bombardment (FAB) mass spectra (MS) were obtained on a VG 7070E-HF instrument. Flash chromatography was performed on Merck Science silica gel 60 (230-400) mesh. Thin layer chromatography (TLC) was performed on analytical Uniplate silica gel GF glass plates (250 mm by 2.5×20 cm²). Preparative TLC was performed on 1.0 or 0.5 mm Analtech silica gel plates. Plates were visualized by UV light, iodine vapor or ninhydrin solution.

7′-{2-[(6-{2-[({(5α,6α)-4,5-Epoxy-3,14-dihydroxy-17-methylmorphin-6-yl}-aminocarbonyl)-methoxy]-acetylamino}-hexylaminocarbonyl)-methoxy]-acetylamino}-naltrindole, MDAN-20, 6. A solution of carboxylic acid 29 (0.904 g, 1.659 mmol, 1.1 eq), DCC (0.342 g, 1.659 mmol, 1.1 eq), and HOBt (0.224 g, 1.659 mmol, 1.1 eq) in DMF (1 mL) was reacted with stirring at room temperature (rt) for 30 min. Amine 26 (0.779 g, 1.508 mmol, 1.0 eq) was dissolved in DMF (2 mL) and added in one portion to the above reaction mixture. This was stirred under N₂ at 60° C. for 24 h. The DCU precipitate was collected via vacuum filtration and the solvent was removed from the filtrate in vacuo. Further purification by flash chromatography (silica gel, D/M/A, 94.5/5/0.5, v/v/v) gave 6 as an off-white solid (43.8%); R_{f} 0.35 (silica gel, D/M/A. 89/10/1. v/v/v); mp 220° C. (decomposes); ¹H NMR (DMSO-d6) δ 10.77 (s, 1H), 9.69 (s, 1H), 8.80 (br s, 1H), 8.04 (t, J=5.1 Hz, 1H), 7.91 (t, J=6.0 Hz, 1H), 7.41 (d, J=8.1 Hz, 1H) 7.15 (d, J=7.2 Hz, 1H), 7.06 (d, J=8.4 Hz, 1H), 6.78 (t, J=7.5 Hz, 1H), 6.46-6.32 (m, 4H), 5.38 (s, 1H), 4.66 (br s, 1H), 4.61 (br s, 1H), 4.32 (d, J=3.3 Hz, 1H), 4.28-4.17 (m, 1H), 4.09 (s, 2H), 3.96 (s, 2H) 3.84 (s, 2H), 3.82 (d, J=3.6 Hz, 2H), 3.15 (d, J=6.6 Hz, 1H), 2.99-2.87 (m, 6H), 2.65-2.50 (m, 4H), 2.41-2.18 (m, 6H), 2.14 (s, 3H), 2.05-1.93 (m, 3H), 1.47-1.39 (m, 2H), 1.33-1.23 (m, 11H), 0.86-0.71 (m, 2H), 0.38-0.34 (m, 2H), 0.02-0.00 (m, 2H); HR-FAB MS m/z 1044.514 M+H)⁺, C₅₇H₆₉N₇O₁₂.H⁺ requires 1044.508; Anal. (C₅₇H₆₉N₇O₁₂.2.75 TFA) calculated: C, 55.43; H, 5.34; N, 7.23. found: C, 55.47; H, 5.31; N, 7.10.

(5α,6α)-6-{2-[(6-{2-[(Methylaminocarbonyl)-methoxy]-acetylamino}-hexylaminocarbonyl)-methoxy]-acetylamino}-4,5-epoxy-3,14-dihydroxy-17-methylmorphinan, MA-20, 13. A solution of carboxylic acid 30 (0.131 g, 0.894 mmol, 1.1 eq), HOBt (0.165 g, 1.22 mmol, 1.5 eq), and DCC (0.184 g, 0.894 mmol, 1.1 eq) in DMF (1.0 mL) was allowed to react at rt with stirring for 10 min. Amine 26 (0.420 g, 0.813 mmol, 1.0 eq) was added in one portion. This mixture was sealed under N₂ and stirred at rt for 48 h. The DCU precipitate was collected via vacuum filtration and the filtrate was added to ethyl ether (100 mL) to facilitate precipitation of the crude product. The solid was collected via vacuum filtration and continuously washed with diethyl ether (50 mL). Further purification via flash chromatography (silica gel, using D/M/A, 93.5/6.0/0.5 (1.5 L) gave 13 as an off white solid (48.2%); R_{f} 0.56 (silica gel, D/M/A. 89/10/1. v/v/v); mp 81° C. (softens), 91° C. (melts); ¹H NMR (DMSO-d6) δ 8.87 (br s, 1H), 8.01-7.93 (m, 3H), 7.50 (d, J=7.8 Hz, 1H), 6.56 (d, J=8.4 Hz, 1H) 6.46 (d, J=8.1 Hz, 1H), 4.77 (br s, 2H), 4.42 (d, J=3.9 Hz, 1H), 4.37-4.29 (m, 1H), 3.96 (s, 2H), 3.93 (d, J=3.3 Hz, 2H), 3.87 (s, 4H), 3.10-2.99 (m, 5H), 2.71 (d, J=6.0 Hz, 1H), 2.62 (d, J=4.5 Hz, 3H), 2.53 (d, J=6.6 Hz, 1H), 2.36 (d, J=6.9 Hz, 1H), 2.27 (s, 3H), 2.17-2.06 (m, 2H), 1.59-1.51 (m, 1H), 1.40-1.23 (m, 1H), 0.93-0.86 (m, 1H); HR-FAB MS m/z 646.345 (M+H)⁺, C₃₂H₄₇N₅O₉.H⁺ requires 646.345; Anal. (C₃₂H₄₇N₅O₉) calculated: C, 59.52; H, 7.34; N, 10.85. found: C, 59.38; H, 7.27; N, 10.71.

7′-{2-[(6-{2-[(Methylaminocarbonyl)-methoxy]-acetylamino}-hexylaminocarbonyl)-methoxy]-acetylamino}-naltrindole, DN-20, 15. A solution of carboxylic acid 30 (0.244 g, 1.658 mmol, 1.1 eq), HOBt (0.224 g, 1.658 mmol, 1.1 eq), and DCC (0.342 g, 1.658 mmol, 1.1 eq) in DMF (2 mL) were incubated with stirring for 60 min. Amine 28 (0.970 g, 1.507 mmol, 1.0 eq) was added to the above reaction mixture. This was sealed under N₂ and stirred at 60° C. (with a reflux condenser present) for 48 h. The DCU precipitate was collected via vacuum filtration and the solvent was removed from the filtrate in vacuo. Further purification by flash chromatography (silica gel with D/M/A, 94.5/5/0.5, v/v/v) gave 15 as a off white solid (40.2%); R_{f} 0.55 (silica gel, D/M/A. 89/10/1. v/v/v); mp 130° C.; ¹H NMR (DMSO-d6) δ 10.77 (br s, 1H), 9.69 (s, 1H), 8.81 (br s, 1H), 8.04 (t, J=5.1 Hz, 1H), 7.89-7.85 (m, 2H) 7.15 (d, J=7.6 Hz, 1H), 7.09 (d, J=7.8 Hz, 1H), 6.78 (t, J=7.5 Hz, 1H), 6.39-6.32 (m, 2H), 5.38 (s, 1H), 4.63 (br s, 1H), 4.09 (s, 2H), 3.95 (s, 2H), 3.75 (s, 4H), 3.16-3.13 (m, 1H), 2.99-2.89 (m, 5H), 2.64-2.53 (m, 3H), 2.49 (d, J=4.5 Hz, 3H), 2.36 (m, 1H), 2.28-2.25 (m, 2H) 2.21-2.14 (m, 1H), 2.05-1.97 (m, 1H), 1.45 (d, J=11.1 Hz, 1H), 1.29 (m, 4H), 1.13 (s, 4H), 0.80-0.72 (m, 1H), 0.41-0.34 (m, 2H), 0.02-0.00 (m, 2H); HR-FAB MS m/z 773.3933 (M+H)⁺, C₄₁H₅₂N₆O₉ requires 772.3796

Animals. Male ICR mice (Harlan, Indianapolis, Ind.) that weighed 25-30 g on arrival were used throughout these studies. The animals were housed in groups of five at 22-23° C. under a 12-12 h light/dark cycle. Both food and water were available ad libitum.

Acute drug administration. Drugs were dissolved in sterile saline (0.9% NaCl). Animals were anesthetized with halothane and drugs were administered with a Hamilton syringe mated to a shortened (3 mm) 27-g needle in a volume of 4 μl by ICV injection into the lateral cerebral ventricle (Haley et al., 1957). The injection site was 1.6 mm lateral and 0.6 mm caudal to bregma.

Antinociceptive testing. Antinociception was evaluated by the radiant heat tail flick assay (D'Amour et al., 1941). Briefly, a beam of light was focused on the mouse tail and the time until the tail flicked was measured. Each animal served as its own control and was used only once. Mice were tested once before injection (control time). After injection, the mice were tested at the time of peak drug response (drug time), as determined by pilot time course studies. The light intensity was adjusted so that control times were between 1.5 and 2.5 s. A 10 second cutoff drug time was set to minimize the risk of tissue damage. Percent maximum possible effect (% MPE) was calculated as follows (Dewey et al., 1970):

$$\frac{Drug Time ( s ) - Control Time ( s )}{10 s - Control Time ( s )} \times 100 \% = \% MPE$$

Graded dose response curves of at least 4 doses with at least 8 mice per dose were generated from the % MPE data. ED50 values with 95% confidence intervals were computed with GraphPad Prism using nonlinear regression methods.

Chronic ICV infusion. Osmotic minipumps (model 1003D, Alzet, Durect Corporation, Cupertino, Calif.) were filled with saline or the drug to be tested. The dose of each drug was twelve times its ED50 (nmol)/hour. The minipumps were connected by a 1.6-1.8 cm length of PE-60 tubing to a 3-mm long cannula (osmotic pump connector cannula, Plastics One, Roanoke, Va.) and primed in sterile saline at 37° C. overnight.

The next day, mice were anesthetized with Avertin (2,2,2-tribromoethanol (370 mg/kg, IP)/tert amyl alcohol (0.16 mg/kg, IP)) before surgery. The scalp was shaved and an incision was made along the midline of the scalp. Hemostats were used to make a pocket under the skin between the shoulder blades. The skull was scraped clean of periosteum so that the cannula pedestal would properly adhere to the skull. A micro drill (Fine Science Tools Inc., Foster City, Calif.) was used to drill a hole approximately 1.6 mm lateral and 0.6 mm caudal to bregma. The minipump was placed between the shoulder blades, the cannula was inserted in the drilled hole into the lateral ventricle, and the cannula pedestal was affixed to the skull with cyanoacrylate glue. The animals were allowed to recover on a heating pad (Fine Science Tools, Foster City, Calif.) and were returned to their cages in the animal facility for three days.

Testing for dependence and tolerance. The development of physical dependence was assessed by quantifying withdrawal jumping observed during precipitated withdrawal (Way et al., 1969) on the fourth day after surgery. Mice were injected with naloxone (1 mg/kg, sc) and placed into Plexiglas cylinders for 10 minutes. During those 10 minutes, vertical jumps were counted as withdrawal signs. Wet-dog shakes were observed in some animals but not recorded.

To test the degree of tolerance developed, the osmotic minipump was removed and the mice were returned to their cages for four hours. The mice were then injected with the test drug into the contralateral lateral cerebral ventricle and antinociception was measured with the tail flick test as described above.

Statistical analyses. ED50 values were considered significantly different when the 95% confidence intervals did not overlap. Significance was accepted at p<0.05.

### Example 2

### Analgesia without Inhibition of GI Transit

One unwanted side effect of treatment with opioids is constipation caused by opioid-induced inhibition of GI transit. Surprisingly, conjugates typically produced less GI transit inhibition than does morphine.

Methods Drugs were administered IV via the tail vein in a volume of 100 μl to male ICR mice. Fifteen minutes later, antinociception was measured by the tail flick assay and charcoal meal (300 μl, oral) was administered by gavage. Thirty minutes after the charcoal meal., the mice were sacrificed by halothane overdose and the distance the charcoal traveled relative to the entire length of the GI tract was compared to the distance traveled in control animals injected with saline. The conjugates tested (MDAN-16, MDAN-19, MDAN-20, and MDAN-21) had linker lengths of 19, 23, 24, and 25 Å.

Results Morphine was fully efficacious in both the TF test and GIT inhibition assay with similar ED50 values (168 (146-178) and 129 (115-142) nmol, respectively). All conjugates exhibited full efficacy in the TF test. However, the maximum GI transit inhibition observed was about 25% at doses which produced more than 80% antinociception. Surprisingly, the MDAN conjugates produced a low amount of GI transit inhibition.

Thus, the conjugates have a therapeutic advantage over traditional opioids for the treatment of both acute and chronic pain as these conjugates produce less GI transit inhibition. As constipation can be caused by opioid-induced inhibition of GI transit, these conjugates will produce less constipation in patients, as compared with constipation caused by other opioid analgesics such as morphine.

### Example 3

### Conditioned Place Preference

Conditioned place preference (CPP) is a technique used to measure the rewarding properties of a drug. Two sides of the CPP apparatus have both visual and tactile differences, so that a mouse can tell the difference between sides. On the first day of testing, the time each mouse spends in either side of the apparatus is measured. For the next three days, the drug is “paired” with one side or the other by injecting the mouse and immediately confining it to that side. On the final day, the amount of time the mouse spends in the drug-paired side is determined and the percent change is calculated. If the percent change is positive, the drug is thought to be rewarding and likely to be abused by humans.

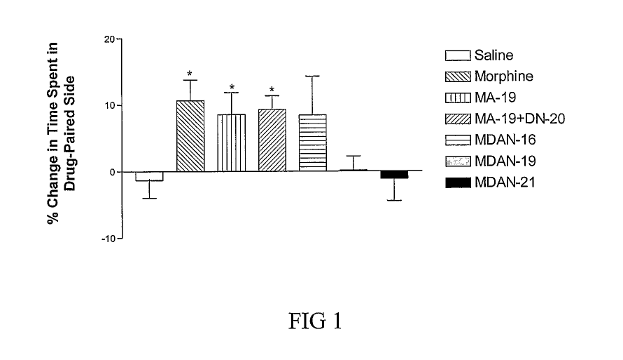

Mice were initially conditioned with morphine at 2× the ED90 analgesic dose, which resulted in the mice exhibiting a conditioned place preference. (FIG. 1) Mice also demonstrated conditioned place preference when treated with 4× the ED90 analgesic dose. Surprisingly, mice treated with either MDAN-19 or MDAN-21 at a dose of 4× the ED90 dose did not display a place preference, whereas results obtained from mice treated with MDAN-16 suggested a place preference, as was shown for morphine.

The results presented herein thus indicate that morphine produced conditioned place preference, but the conjugate MDAN-21, which does not cause tolerance or physical dependence, also does not produce conditioned place preference. These results indicate that the conjugates have a lowered reinforcing effect, as compared to morphine, thereby indicating that the conjugates will have low potential for causing addiction.

Methods The CPP apparatus used was a plastic box, approximately 12 inches×6 inches×6 inches (1/w/h). One half of the box was transparent with a scored or textured floor and the other half of the box had blue vertical stripes with a smooth floor. The box was divided such that the mice could go from one side to the other through an opening or be confined to one side.

Preconditioning On day 1, each mouse was allowed to explore both sides of the box for 15 minutes to expose them to the novel environment. On day 2, each mouse was placed into the box for 15 minutes and the amount of time the mouse spent in each side of the box was recorded. Mice that spend more than 9 minutes in one side of the box are excluded (two of 32 mice were excluded).

Conditioning On day 3, each mouse was given two sets of IV injections. First, the mice were injected with saline and randomly confined to one side of the box for 30 minutes. The mice were then injected with the drug (or saline, for control mice) and confined to the other side of the box (“drug-paired side”) for 30 minutes. This conditioning paradigm was repeated for three days.

Postconditioning On day 6, the amount of time the mouse spent in each side of the box was recorded. For the results, the amount of time the mouse spent in the drug-paired side during preconditioning was subtracted from the time spent in the drug-paired side during post-conditioning to reach a “score”: Score=Postconditioning time in drug-paired side-Preconditioning time spent in drug-paired side.

FIG. 1 depicts the percent change in time spent in the drug-paired side. The asterisks in the graph indicate significant difference from saline (p<0.05).

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

All publications, patents and patent applications cited herein are herein incorporated by reference.

### DOCUMENTS CITED

Abdelhamid et al., Journal of Pharmacology and Experimental Therapeutics, 1991, 258, 299-303.
Ananthan et al., Journal of Medicinal Chemistry, 1999, 42, 3527-3538.
Bardo et al., Psychopharmacology, 2000, 153, 31-43.
Costa et al., Life Sciences, 1982, 31, 1625-1632.
Costa et al., Biochemical Pharmacology, 1985, 34, 25-30.
D'Amour et al., J Pharmacol Exp Ther, 1941, 72:74-79.
Daniels et al., “Delta-Selective Ligands Related to Naltrindole”, in “The Delta Receptor”, Chang et al., Eds. Marcel Dekker, Chapter 9, pages 139-158 (2003).
Dewey et al., J Pharmacol Exp Ther, 1970, 175: 435-442.
Erez et al., Journal of Medicinal Chemistry, 1982, 25, 847-849.
Foye's Principles of Medicinal Chemistry, 5th Ed., D. A. Williams and T. L. Lemke, Eds, Lippencott, Williams, and Wilkins.
George et al., Journal of Biological Chemistry, 2000, 275, 26128-26135.
Gomes et al., The Journal of Neuroscience, 2000, 20, 1-5.
Gomori et al., Am J Physiol Endocrinol Metab, 2003, 284, E583-8.
Haley et al., British Journal of Pharmacology, 1957 12, 12-15.
Hammond, in Advances in Pain Research and Therapy:, eds. Chapman, C. R. & Loeser, J. D. (Raven, New York), 1989 69-91.
Hazum et al., Biochemical and Biophysical Research Communications, 1982, 104, 347-353.
Heyman et al., Progress in Opioid Research, 1989, 75, 467-470.
Ho et al., Journal of Pharmacology and Experimental Therapeutics, 1972, 182, 155-165.
Ji et al., J. Neurosci., 1995, 15(12), 8156-8166.
Kest et al., Brain Research Bulletin, 1996, 39, 185-189.
Kivell et al., Brain Res Dev Brain Res, 2004, 149(1), 9-19.
Mashiko et al., Endocrinology, 2003, 144, 1793-801.
Miyamoto et al., Journal of Pharmacology and Experimental Therapeutics, 1993, 264, 1141-1145.
Miyamoto et al., Journal of Pharmacology and Experimental Therapeutics, 1994, 270, 37-39.
Ohkawa et al., Journal of Medicinal Chemistry, 1997, 40, 1720-1725.
Ohkawa et al., Journal of Medicinal Chemistry, 1998, 41, 4177-4180.
Portoghese et al., Life Sciences, 1982, 31, 1283-1286.
Portoghese et al., Journal of Medicinal Chemistry, 1985, 28, 1140-1141.
Portoghese et al., Journal of Medicinal Chemistry, 1986, 29, 1650-1653.
Portoghese et al., Journal of Medicinal Chemistry, 1986, 29, 1855-1861.
Portoghese et al., Journal of Medicinal Chemistry, 1987, 30, 1991-1994.
Portoghese et al., Journal of Medicinal Chemistry, 1988, 31, 281.
Portoghese et al., Journal of Medicinal Chemistry, 1994, 37, 579-585.
Sánchez-Blázquez et al., Journal of Pharmacology and Experimental Therapeutics, 1997, 280, 1423-1431.
Sasaki-Yagi et al., International Journal of Peptide and Protein Research, 1991, 38, 378-384.
Schiller et al., Journal of Medicinal Chemistry, 1999, 42, 3520-3526.
Shimohigashi et al., Nature, 1982, 297, 333-335.
Takemori et al., European Journal of Pharmacology, 1990, 186, 285-288.
Van der Kooy et al., Brain Research, 1982, 243, 107-117.
Vaught et al., Journal of Pharmacology and Experimental Therapeutics, 1979, 208, 86-94.
Vaught et al., Life Sciences, 1982, 30, 1443-1455.
Wang et al., J Neurosci, 2001, 21(9), 3242-3250.
Way, Annals of the New York Academy of Science, 1978, 311, 61-68.
Way et al., J Pharmacol Exp Ther, 1969, 167:1-8.
Wells et al., Journal of Pharmacology and Experimental Therapeutics, 2001, 297, 597-605.
Wu et al., Neurosci Lett, 2004, 365, 157-61.
Zhu et al., Neuron, 1999 24, 243-252.

1-4. (canceled)

5. The method of claim 60, wherein administration of the conjugate causes less inhibition of gastrointestinal (GI) transit than is caused by administration of a similar effective dosage of morphine to a patient.

6. The method of claim 60, wherein administration of the conjugate causes less constipation than is caused by administration of a similar effective dosage of morphine to a patient.

7. (canceled)

8. The method of claim 60, wherein administration of the conjugate causes less dependence than is caused by administration of a similar effective dosage of morphine to a patient.

9. The method of claim 60, wherein the administration of the conjugate causes less tolerance than is caused by administration of a similar effective dosage of morphine to a patient.

10. The method of claim 60, wherein the conjugate has the formula:
R₁—X₁—R₂
wherein
R₁ is a mu opioid receptor agonist;
R₂ is a delta opioid receptor antagonist; and
X₁ is a linker.

11. The method of claim 10, wherein X₁ comprises an amino acid.

12. The method of claim 10, wherein X₁ comprises a peptide.

13. The method of claim 10, wherein X₁ is a divalent, branched or unbranched, saturated or unsaturated, hydrocarbon chain, having from 10-30 carbon atoms in the chain, wherein one or more of the carbon atoms in the chain is optionally replaced by (—O—) or (—NH—).

14. The method use of claim 10, wherein X₁ is a divalent, branched or unbranched, saturated or unsaturated, hydrocarbon chain, having from 10-30 carbon atoms in the chain, wherein one or more of the carbon atoms in the chain is optionally replaced by (—O—) or (—NH—), and wherein the chain is optionally substituted on at least one carbon, —O— or —NH— with one or more substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

15. The method of claim 10, wherein X₁ is a chain 18-24 atoms in length.

16. The method of claim 10, wherein X₁ comprises a central diamine moiety having adjacent diglycolic acid molecules.

17. The method of claim 10, wherein X₁ comprises at least one methylene.

18. The method of claim 10, wherein R₁ is oxymorphone, α-oxymorphamine, a benzomorphan, etonitazine, fentanyl, or a compound of formula 100, 101, 102, 103, 104, or a derivative thereof.

19. The method of claim 10, wherein R₂ is naltrindole or a compound of formula 201, 202, or 203, or a derivative thereof.

20. The method of claim 60, wherein the conjugate has the following formula: 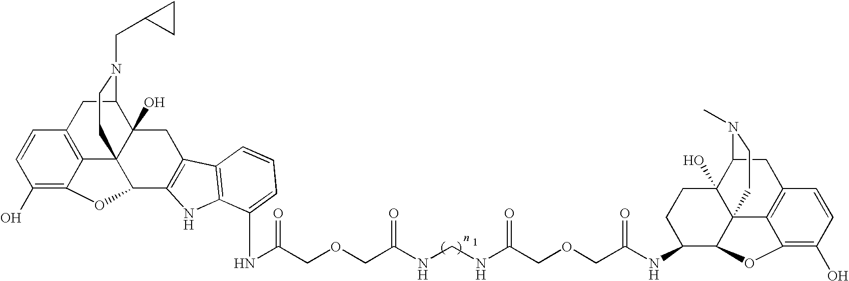
wherein n₁ is 1, 2, 3, 4, 5, 6, 7, 8, 9, or 10.

21. The method of claim 20, wherein n₁ is 5, 6, or 7.

22. The method of claim 60, wherein administration of the conjugate is intravenous.

23. The method of claim 60, wherein the conjugate is administered in combination with at least one additional therapeutic agent.

24. A pharmaceutical composition comprising:
a conjugate that comprises a mu opioid receptor agonist linked via a linker to a delta opioid receptor antagonist; and
a pharmaceutically acceptable carrier other than saline;
which composition is formulated for IV administration.

25. A pharmaceutical composition comprising:
a conjugate that comprises a mu opioid receptor agonist linked via a linker to a delta opioid receptor antagonist; and
a pharmaceutically acceptable carrier that comprises saline and at least one other pharmaceutically acceptable carrier;
which composition is formulated for intravenous administration.

26. The composition of claim 24, wherein the conjugate has the formula:
R₁—X₁—R₂
wherein
R₁ is a mu opioid receptor agonist;
R₂ is a delta opioid receptor antagonist; and
X₁ is a linker.

27. The pharmaceutical composition of claim 26, wherein X₁ comprises an amino acid.

28. The pharmaceutical composition of claim 26, wherein X₁ comprises a peptide.

29. The pharmaceutical composition of claim 26, wherein X₁ is a divalent, branched or unbranched, saturated or unsaturated, hydrocarbon chain, having from 10-30 carbon atoms, wherein one or more of the carbon atoms is optionally replaced by (—O—) or (—NH—).

30. The pharmaceutical composition of claim 26, wherein X₁ is a divalent, branched or unbranched, saturated or unsaturated, hydrocarbon chain, having from 10-30 carbon atoms, wherein one or more of the carbon atoms is optionally replaced by (—O—) or (—NH—), and wherein the chain is optionally substituted on at least one carbon, —O— or —NH— with one or more substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

31. The pharmaceutical composition of claim 26, wherein X₁ is a chain 18-24 atoms in length.

32. The pharmaceutical composition of claim 26, wherein X₁ comprises a central diamine moiety having adjacent diglycolic acid molecules.

33. The pharmaceutical composition of claim 26, wherein X₁ comprises at least one methylene.

34. The pharmaceutical composition of claim 26, wherein R₁ is oxymorphone, oxymorphone, α-oxymorphamine, a benzomorphan, etonitazine, fentanyl, or a compound of formula 100, 101, 102, 103, 104, or a derivative thereof.

35. The pharmaceutical composition of claim 26, wherein R₂ is naltrindole or a compound of formula 201, 202, or 203, or a derivative thereof.

36. The pharmaceutical composition of claim 24, wherein the conjugate has the following formula: 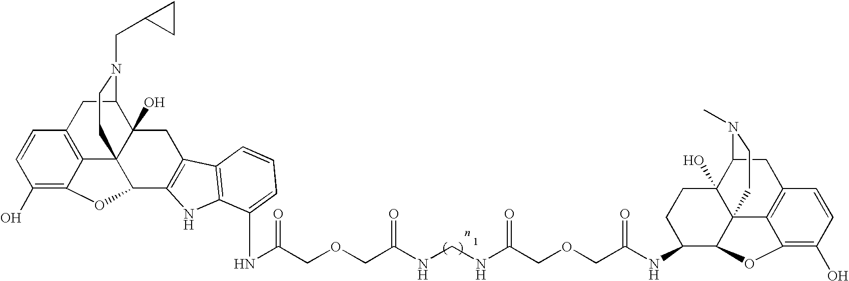
wherein n₁ is 1, 2, 3, 4, 5, 6, 7, 8, 9, or 10.

37. The pharmaceutical composition of claim 36, wherein n₁ is 5, 6, or 7.

38. A conjugate having the formula:
R₁—X₁—R₂
wherein
R₁ is a mu opioid receptor agonist;
R₂ is a delta opioid receptor antagonist; and
X₁ is a linker, provided that the conjugate is not a conjugate of the formula: 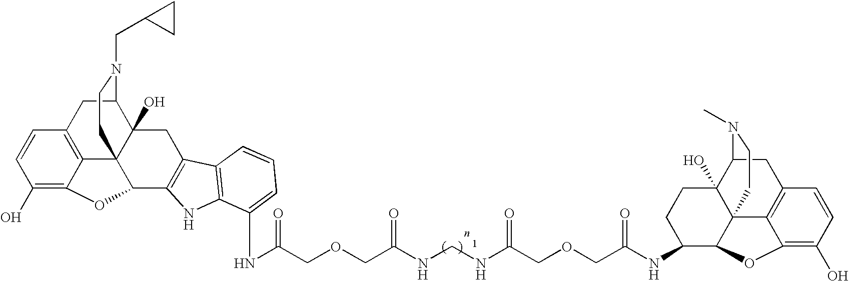
wherein n₁ is 2, 3, 4, 5, 6, or 7.

39. A conjugate having the formula:
R₁—X₁—R₂
wherein
R₁ is a mu opioid receptor agonist that is not α-oxymorphamine;
R₂ is a delta opioid receptor antagonist; and
X₁ is a linker.

40. The conjugate of claim 38, wherein R₁ is a benzomorphan, etonitazine, fentanyl, or a compound of formula 100, 101, 102, 103, 104, or a derivative thereof.

41. A conjugate having the formula:
R₁—X₁—R₂
wherein
R₁ is a mu opioid receptor agonist;
R₂ is a delta opioid receptor antagonist that is not naltrindole; and
X₁ is a linker.

42. The conjugate of claim 38, wherein R₂ is derivative or naltrindole or a compound of formula 201, 202, or 203, or a derivative thereof.

43. A conjugate having the formula:
R₁—X₁—R₂
wherein
R₁ is a mu opioid receptor agonist;
R₂ is a delta opioid receptor antagonist; and
X₁ is a linker that is not 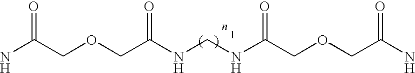
wherein n₁ is 2, 3, 4, 5, 6, or 7.

44. The conjugate of claim 38, wherein X₁ comprises an amino acid.

45. The conjugate of claim 38, wherein X₁ comprises a peptide.

46. The conjugate of claim 38, wherein X₁ is a divalent, branched or unbranched, saturated or unsaturated, hydrocarbon chain, having from 10-30 carbon atoms, wherein one or more of the carbon atoms is optionally replaced by (—O—) or (—NH—).

47. The conjugate of claim 38, wherein X₁ is a divalent, branched or unbranched, saturated or unsaturated, hydrocarbon chain, having from 10-30 carbon atoms, wherein one or more of the carbon atoms is optionally replaced by (—O—) or (—NH—), and wherein the chain is optionally substituted on at least one carbon, —O— or —NH— with one or more substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

48. The conjugate of claim 38, wherein X₁ is a chain 18-24 atoms in length.

49. The conjugate of claim 38, wherein X₁ comprises a central diamine moiety having adjacent diglycolic acid molecules.

50. The conjugate of claim 38, wherein X₁ comprises at least one methylene.

51. The conjugate of claim 41, wherein R₁ is oxymorphone, α-oxymorphamine, a benzomorphan, etonitazine, fentanyl, or a compound of formula 100, 101, 102, 103, 104, or a derivative thereof.

52. The conjugate of claim 39, wherein R₂ is naltrindole or a compound of formula 201, 202, or 203, or a derivative thereof.

53. A pharmaceutical composition comprising a pharmaceutically acceptable excipient and a conjugate as described in claim 38.

54. A unit dosage form comprising a conjugate as described in claim 38 and a pharmaceutically acceptable excipient.

55-59. (canceled)

60. A method for producing analgesia in a patient, comprising administering to the patient an effective amount of a conjugate that comprises a mu opioid receptor agonist linked via a linker to a delta opioid receptor antagonist, which administration is to a location outside the central nervous system of the patient.

61. A method for producing analgesia while causing less inhibition of gastrointestinal (GI) transit than is caused by administration of a similar effective dosage of morphine in a patient, comprising administering to the patient an effective amount of a conjugate that comprises a mu opioid receptor agonist linked via a linker to a delta opioid receptor antagonist effective to cause analgesia while causing less inhibition of GI transit than is caused by administration of a similar effective dosage of morphine to a patient.

62. A method for producing analgesia while causing less dependence than is caused by administration of a similar effective dosage of morphine in a patient, comprising administering to the patient an effective amount of a conjugate that comprises a mu opioid receptor agonist linked via a linker to a delta opioid receptor antagonist effective to cause analgesia while causing less dependence than is caused by administration of a similar effective dosage of morphine to a patient.

63. A method for producing analgesia while causing less tolerance than is caused by administration of a similar effective dosage of morphine in a patient, comprising administering to the patient an effective amount of a conjugate that comprises a mu opioid receptor agonist linked via a linker to a delta opioid receptor antagonist effective to cause analgesia while causing less tolerance than is caused by administration of a similar effective dosage of morphine to a patient.

64. (canceled)
